(19) United States
(12) Patent Application Publication
(10) Pub. No.: US 2019/0202759 A1
(43) Pub. Date: Jul. 4, 2019

(54) PROCESSES FOR THE DEHYDROCHLORINATION OF A CHLORINATED ALKANE

(71) Applicant: BLUE CUBE IP LLC, Clayton, MO (US)

(72) Inventors: John D. Myers, Clayton, MO (US); Max M. Tirtowidjojo, Clayton, MO (US)

(21) Appl. No.: 16/331,726

(22) PCT Filed: Sep. 5, 2017
(86) PCT No.: PCT/US2017/050038

§ 371 Date: Mar. 8, 2019

Related U.S. Application Data
(60) Provisional application No. 62/385,759, filed on Sep. 9, 2016.

(51) Int. Cl. C07C 17/25; C07C 17/04; B01J 31/02

(52) U.S. Cl. CPC C07C 17/25; C07C 21/10; B01J 31/0239; C07C 17/04

(57) ABSTRACT

The present invention provides a process for the dehydrochlorination of a chlorinated alkane to produce a chlorinated alkene. In particular, the processes comprise contacting a chlorinated alkane, a base, and a phase transfer catalyst.

### FIELD OF THE INVENTION

The present disclosure generally relates to processes for the dehydrochlorination of a chlorinated alkane.

### BACKGROUND OF THE INVENTION

Chlorinated alkenes are useful intermediates for many products including agricultural products, pharmaceuticals, cleaning solvents, blowing agent, gums, silicons, and refrigerants. A general preparation of chloroalkenes is using a dehydrochlorination process. The most widely used of the dehydrochlorination processes utilize a Lewis acid catalyst, such as FeCl₃ or AlCl₃. In each of these cases, the catalyst is not complexed with a ligand since this ligand complexation can reduce the rate and yield of the chlorinated alkene.

The chlorinated alkanes useful in the preparation of some chlorinated alkenes are produced through the telomerization of carbon tetrachloride (Tet), ethylene or vinyl chloride and a catalyst system comprising metallic iron, tributylphosphate (TBP), and FeCl₃ producing a tetrachloropropane or pentachloropropane. The active catalyst in this telomerization process is a Fe-TBP catalyst where TBP is the coordinating ligand. At the completion of the process, the TBP must be removed often using distillation from the reactor product prior to the dehydrochlorination process. If the TBP is not removed, the activity of the dehydrochlorination catalyst is inhibited, the process produces heavy by-products, and yields decrease in the subsequent dehydrochlorination process.

Another process for dehydrochlorination of a chlorinated alkane utilizes a base, such as sodium hydroxide. These processes are known yet these processes utilize purified tetrachloropropanes, instead of crude or unpurified tetrachloropropanes. Additionally, these processes are silent on removing the iron from the previous telomerization reaction in the dehydrochlorination process and provide no suggestion on recycling valuable materials to other processes.

Developing a dehydrochlorination process which utilizes crude chlorinated alkanes, allows for recovery and recycle of TBP, reduces the byproduct formation, reduces or eliminates the need for distillation between the telomerization and dehydrochlorination processes, and utilizes an inexpensive product from the chloroalkali process will provide highly efficient, cost effective, and robust process.

### SUMMARY OF THE INVENTION

In one aspect, disclosed herein are processes for dehydrochlorinating a chlorinated alkane in a liquid phase using an aqueous phase comprising an inorganic base derived from the chloroalkali process. Once the desire chlorinated alkene is prepared, the reactor contents are transferred into a separator where the chlorinated alkene is isolated.

In another aspect, disclosed herein are processes for preparing trichloropropene isomers from an unpurified stream comprising 1,1,1,3-tetrachloropropane using an aqueous phase comprising an inorganic base derived from the chloroalkali process in liquid phase. Once the desired trichloropropene isomers are prepared, the reactor contents are transferred to a separator where the trichloropropene isomers are isolated, and valuable components such as iron hydroxide, TBP, and other components may be recycled to other processes.

Other features and iterations of the invention are described in more detail below.

### DETAIL DESCRIPTION OF FIGURES

The following figures illustrate non-limiting embodiments of the present invention wherein:

FIG. 1 is a graphical representation showing the percent (%) conversion of 1,1,1,3-tetrachloropropane (250FB) and the selectivity to the desired 1,1,3- and 3,3,3-trichloropropenes (113e and 333e) from the dehydrochlorination of purified 1,1,1,3-tetrachloropropane using aqueous NaOH.

FIG. 2 is a graphical representation showing the percent (%) conversion of 1,1,1,3-tetrachloropropane (250FB) and the selectivity to the desired 1,1,3- and 3,3,3-trichloropropenes (113e and 333e) from the dehydrochlorination of unpurified 1,1,1,3-tetrachloropropane using aqueous NaOH.

FIG. 3 is a graphical representation showing the percent (%) conversion of 1,1,1,3-tetrachloropropane (250FB) and the selectivity to the desired 1,1,3- and 3,3,3-trichloropropenes (113e and 333e) from the dehydrochlorination of crude 1,1,1,3-tetrachloropropane using aqueous NaOH. This figure further show Aliquat 336 that was fed to the telomerization reaction remained active in dehydrochlorination reaction and the impurities did not adversely affect the reaction.

### DETAILED DESCRIPTION OF THE INVENTION

In one aspect, the processes for preparing chlorinated alkenes comprise contacting a chlorinated alkane, an aqueous phase comprising an inorganic base derived from the chloroalkali process, and a phase transfer catalyst in liquid phase. The process may be termed a dehydrochlorination reaction. The contents from the process may be further purified. The trichloropropenes, either purified or unpurified, may be utilized in further processes.

### (I) Process for Preparing Chlorinated Alkenes

The process for preparing chlorinated alkenes comprises contacting a liquid chlorinated alkane or a liquid chlorinated alkane process stream, an aqueous phase comprising an inorganic base derived from the chloroalkai process, and a phase transfer catalyst under process conditions to enable the preparation of an effective high yield of the chloroalkene product.

As compared to other dehdrochlorination processes, it was unexpectedly found utilizing an aqueous base from the chloroalkali process with purified or unpurified tetrachloropropanes from the telomerization process provides high selectivity and conversion of the trichloropropenes without excess amounts of heavy byproducts. Additionally, the process allows for recovery of valuable catalysts which may be utilized in other processes.

(a) Chlorinated Alkane

The chlorinated alkane useful in this process may be a tetrachloropropane. Tetrachloropropanes are typically produced by the telomerization of carbon tetrachloride (Tet) and ethylene in the presence of a catalyst system comprising metallic iron, FeCl₃, and tributyl phosphate (TBP) or phosphites. The tetrachloropropanes from the telomerization process may contain a soluble Fe-TBP complex, unreacted Tet, dissolved ethylene, and heavy byproducts such as tetrachloropentane isomers. In a preferred embodiment, the tetrachloropropane is 1,1,1,3-tetrachloropropane, also known as 250FB.

The tetrachloropropane may be used directly from the telomerization process as a process stream, or partially purified, by means known to the skilled artisan, such as distillation, before the dehydrochlorination process. In various embodiments, the partially purified tetrachloropropane may comprise lighter by products, such as Tet and ethylene. In other embodiments, the partially purified tetrachloropropane may contain a soluble Fe-TBP catalyst, higher boiling point chlorocarbons, and heavier by products. In each of these cases, the tetrachloropropane may be used as the limiting reagent in the dehydrochlorination process.

Generally, the tetrachloropropane useful in the process may have a purity greater than 10 wt %. In various embodiments, the purity of the tetrachloropropane may have a purity greater than 10 wt %, greater than 30 wt %, greater than 50 wt %, greater than 75 wt %, greater than 90 wt %, greater than 95 wt %, or greater than 99 wt %.

(b) Phase Transfer Catalyst

A wide variety of phase transfer catalyst may be used in the dehydrochlorination of chlorinated alkanes to produce chlorinated alkenes. Non-limiting examples of phase transfer catalysts may be quaternary ammonium salts, phosphonium salts, pyridinium salts, or combinations thereof. In some embodiments, the phase transfer catalyst may be a quaternary ammonium salt. Non-limiting examples of suitable salts may be chloride, bromide, iodide, or acetate. Non-limiting examples quaternary ammonium salts may be trioctylmethylammonium chloride (Aliquat 336), trioctylmethylammonium bromide, dioctyldimethylammonium chloride, dioctyldimethylammonium bromide, Arquad 2HT-75, benzyldimethyldecylammonium chloride, benzyldimethyldecylammonium bromide, benzyldimethyldecylammonium iodide, benzyldimethyltetradecylammonium chloride, dimethyldioctadecylammonium chloride, dodecyltrimethylammonium chloride, tetrabutylammonium chloride, tetrabutylammonium bromide, tetrabutylammonium iodide, tetrabutylammonium acetate, tetrahexylammonium chloride, tetraoctylammonium chloride, tridodecylmethylammonium chloride, tetraethylammonium chloride, tetraethylammonium bromide, tetraethylammonium iodide, or combinations thereof. Non-limiting examples of phosphonium salts may be tetrabutylphosphonium bromide, dimethyldiphenyl phosphonium iodide, tetramethylphosphonium chloride, tetraphenylphosphonium bromide, trihexyltetradecylphosphonium chloride, or combinations thereof. Non-limiting examples of pyridinium salts may be cetylpyridinium chloride, hexadecylpyridinium bromide, hexadecylpyridinium chloride monohydrate, or combinations thereof. In a preferred embodiment, the phase transfer catalyst may be trioctylmethylammonium chloride (Aliquat 336).

Generally, the amount of the phase transfer catalyst may range from 0.05 wt % to about 5.0 wt % based on the total weight of the components. In various embodiments, the amount of the phase transfer catalyst may range from 0.05 wt % to about 5 wt %, from 0.1 wt % to 2.5 wt %, from 0.3 wt % to about 1 wt %, or from 0.4 wt % to about 0.7 wt %.

(c) Aqueous Phase Comprising an Inorganic Base

The dehydrochlorination process utilizes an aqueous phase comprising an inorganic base which is produced from the chloroalkali process. The aqueous base may contain an inorganic chloride salt.

The inorganic base may be an alkali or alkali earth metal hydroxide. Non-limiting examples of these alkali or alkali earth hydroxides may be LiOH, NaOH, KOH, Ba(OH)₂, or Ca(OH)₂. In a preferred embodiment, the alkali or alkali earth metal hydroxide may be NaOH.

The inorganic chloride salt may be any alkali or alkali earth metal chloride salt. Non-limiting examples of these alkali or alkali earth metal salt chloride salts may be selected from a group consisting of lithium chloride, sodium chloride, potassium chloride, barium chloride, calcium chloride, or combinations thereof. In a preferred embodiment, the chloride salt may be sodium chloride.

In another embodiment, an aqueous phase comprises a mixture of NaOH and at least one chloride salt which was produced from the chloroalkali process through the electrolysis of sodium chloride in a diaphragm cell. Generally, the concentration of the sodium hydroxide may be less than 20 wt %. In various embodiments, the concentration of sodium hydroxide may be less than 20 wt %, less than 15 wt %, less than 10 wt %, less than 8 wt %, less than 5 wt %, less than 2 wt %, and less than 1 wt %. Additionally, the concentration of the sodium chloride is less than 26 wt %. In various embodiments, the concentration of sodium chloride is less than 26 wt %, less than 20 wt %, less than 15 wt %, less than 10 wt %, less than 8 wt %, less than 5 wt %, less than 2 wt %, and less than 1 wt %.

In general, the mole ratio of the base(s) to the chlorinated alkane may range from 0.1:1.0 to about 2.0:1.0. In various embodiments, the mole ratio of the base(s) to the chlorinated alkane may range from 0.1:1.0 to about 2.0:1.0, from 1.0:1.0 to about 1.75:1.0, or from 1.05:1.0 to about 1.3:1.0.

(d) Reaction Conditions

In general, the dehydrochlorination process for producing a chlorinated alkene includes carrying out the dehydrochlorination reaction in liquid phase at process conditions to enable the preparation of an effective high yield of the chloroalkene product.

The process commences by contacting the tetrachloropropane, either partially purified or unpurified, an aqueous phase comprising an inorganic base, and a phase transfer catalyst in liquid phase. All the components of the process are typically mixed at a temperature enabling the preparation of effective high yield of the chloroalkene product. In a preferred embodiment, the tetrachloroalkane and phase transfer catalyst are mixed at a specified temperature to produce a solution, then the aqueous phase is added, either incrementally or continuously.

The temperature of the process can and will vary depending on purity of the tetrachloroalkane, the phase transfer catalyst, the base, and the concentration of the base. Generally, the temperature of the process may be generally from 45° C. to about 100° C. In various embodiments, the temperature of the process may be generally from 45° C. to about 100° C., from 50° C. to about 80° C., or from 60° C. to 70° C.

In general, the pressure of the process may range from 0 psig to about 200 psig. In various embodiments, the pressure of the process may range from 0 psig to about 200 psig, from 10 psig to about 100 psig, from 20 psig to about 50 psig, or from 30 psig to about 40 psig. In a preferred embodiment, the pressure of the process may be about atmospheric pressure and the process may be conducted under an inert atmosphere such as nitrogen, argon, or helium.

Generally, the reaction is allowed to proceed for a sufficient period of time until the reaction is complete, as determined by any method known to one skilled in the art, such as chromatography (e.g., GC). The duration of the reaction may range from about 5 minutes to about 8 hours. In some embodiments, the duration of the reaction may range from about 5 minutes to about 7 hours, from about 30 minutes to about 6 hours, from about 2 hours to about 5 hours, or from about 3 hours to about 4.

As appreciated by the skilled artisan, the above process may be run in a batch mode or a continuous mode. In another embodiment, the process in continuous modes may be stirred in various methods to improve the mixing of the biphasic system as appreciated by the skilled artisan. One preferred method for ensuring the biphasic contents of the reactor are adequately mixed may be utilizing a jet stirred reactor which mixes the contents of the reactor without an impeller. In this jet stirred reactor system, the liquid materials comprising of internal recycle and fresh feed are transported vertically or tangentially through the reactor by means of an external pump. A portion of the reaction product is recycled back to the reactor while the rest is removed from the reaction system into the purification step.

The tetrachloropropane fed to the above described process may be converted to the trichloropropene isomers in at least 50% conversion. In various embodiments, the conversion of tetrachloropropane to the trichloropropene isomers may be at least 50%, at least 60%, at least 75%, at least 85%, at least 95%, and at least 99%.

The selectivity to the desired trichloropropenes can and will vary depending on the reaction conditions, base, the purity level of the tetrachloropropane utilized, and the trichloropropenes produced. Generally, the selectivity to the trichloropropenes may be greater than 50%. In various embodiments, the selectivity to the desired trichloropropenes may be greater than 50%, greater than 60%, greater than 70%, greater than 80%, or greater than 90%. In preferred embodiments, the selectivity to the desired trichloropropenes may range from 95% to 99%.

### (ii) Separating Chlorinated Alkene Products.

The next step in the process comprises separating purified chlorinated alkenes from the contents of the reactor comprising the trichloropropenes, iron hydroxide, salt, water, TBP, Tet, ethylene, lighter by products, heavier by products, and unreacted chloropropane starting material. Alternatively, the next step is to utilize the contents of the reactor comprising the trichloropropene, iron hydroxide, salt, water, TBP, Tet, ethylene, lighter by products, heavier by products, and unreacted chloropropane starting material in another process. In a preferred embodiment, the chlorinated alkene product may comprise a mixture of 1,1,3-trichloropropene, 3,3,3-trichloropropene, and 1,2,3-trichloropropene.

The separation process commences by transferring the reactor contents into a separator or multiple separators. As appreciated by the skilled artisan, many separation techniques may be useful. Non-limiting examples of separation techniques may be decantation, settling, filtration, separation, centrifugation, thin film evaporation, simple distillation, vacuum distillation, fractional distillation, or a combination thereof. The distillations may comprise at least one theoretical plate.

Depending on the quality and purity of the tetrachloropropane, various separation processes may be employed in various orders.

During the dehydrochlorination process, the catalyst (Fe-TBP) is hydrolyzed to form iron hydroxide and TBP. The contents of the reactor are transferred to a separation device where the aqueous phase, containing all or part of the iron hydroxide can be separated from the organic phase of the reactor contents by a phase separation vessel wherein the aqueous phase can be withdrawn from near or the top and the organic phase can be withdrawn from near the bottom of said vessel. Then, the aqueous phase may be further separated to remove iron hydroxide by filtration, centrifugation, or settling. The iron hydroxide may be recycled to another process. Alternatively, the aqueous phase including the iron hydroxide may be sent to a waste treatment process. The organic phase, removed from the phase separator, may be distilled to produce purified trichloropropenes, a stream comprising the light by products, water, and a stream comprising higher boiling point chlorocarbons, phase transfer catalyst, TBP, heavier by products, and combinations thereof. The distilled light by products may be recycled to another process. The distilled TBP, higher boiling point chlorocarbons, phase transfer catalyst, and heavier by products may be recycled to another process. Recovered phase transfer catalyst may also be utilized in other processes including another dehydrochlorination as described above. A portion of the high boiling point chlorocarbons, phase transfer catalyst, heavier by products, and combinations thereof may be recycled to the process to prepare the chlorinated alkane starting material. A portion of the high boiling point chlorocarbons, phase transfer catalyst, heavier byproducts, and combinations thereof may be subjected to further separations or may be purged from the system to prevent excessive accumulation of high boiling point chlorocarbons and heavier byproducts.

The product stream from the separator comprising the chlorinated alkene produced in the process may have a yield of at least about 10%. In various embodiments, the product stream comprising chlorinated alkene produced in the process may have a yield of at least about 20%, at least about 50%, at least about 70%, at least about 75%, at least about 80%, at least about 85%, at least about 90%, at least about 95%, or at least about 99%.

### (III) Utilizing Chlorinated Alkene Products.

The trichloropropenes (purified or unpurified) may be utilized in further processes. Chlorination with SO₂Cl₂, Cl₂, or a combination thereof would produce 1,1,1,2,3-pentachloropropane. Dehydrochlorination of the 1,1,1,2,3-pentachloropropane using base, catalysts, or combinations thereof would yield 1,1,2,3-tetrachloropropene.

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

Having described the invention in detail, it will be apparent that modifications and variations are possible without departing from the scope of the invention defined in the appended claims.

### EXAMPLES

The following examples illustrate various embodiments of the invention.

### Example 1: Dehydrochlorination of 250FB (Baseline Case)

12.4 grams of pure 250FB and 0.073 g trioctyl methyl ammonium chloride (Aliquat 336) phase transfer catalyst was added to a 50 cc reaction flask. The flask temperature was raised to 65-70° C. The temperature was maintained at 65-70 C using an electric heater. A solution of 8.8% NaOH and 16% NaCl was added incrementally over a time period of about 1 hour. The total amount of NaOH/NaCl/H₂O solution added was 32.1 g. Agitation of the aqueous and organic phases was achieved with a magnetic stirring bar. After all of the NaOH/NaCl/H₂O solution was added, the reaction was stirred for an additional 2 hours. Periodic samples of the organic phase were taken by stopping stirring and drawing samples into a syringe from the bottom of the flask. The samples were analyzed by gas chromatography.

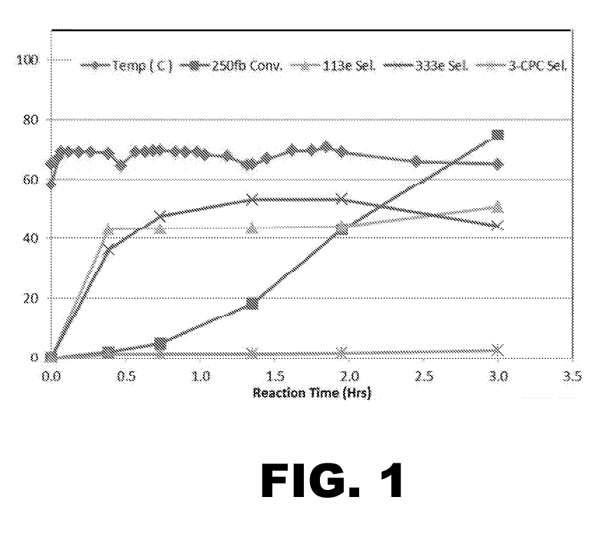

FIG. 1 shows that the conversion of 250FB was 75%. The selectivities to the desired 113e and 333e were both in the range from 45-50%. The selectivity to the primary by-product (labeled 3-CPC, which was probably a hydroxychloropropane or propionyl chloride) was 2.5%.

### Example 2: Dehydrochlorination of 250FB

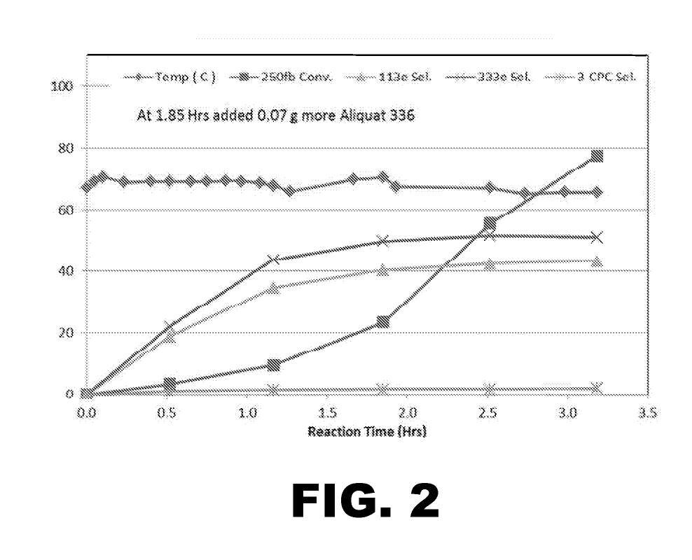

Example 1 was repeated using 10.3 g crude 250FB from a telomerization reaction of Tet and ethylene. The crude feed contained residual Tet, ethylene, by-product tetrachloropentanes, other minor by-products, and an Fe-TBP catalyst complex. Initially, 0.05 g Aliquat 336 was added. After 1.85 hours, 0.07 g of additional Aliquat 336 was added. The amount of NaOH/NaCl/H₂O solution was in 1.05 molar excess of NaOH versus 250FB. After 3.2 hours, the reaction mixture was cooled and the aqueous phase was separated and allowed to settle. The clarified aqueous phase did not contain any detectable iron. FIG. 2 shows that conversion and selectivity similar to using purified 250FB feed were obtained. The impurities did not adversely affect the reaction.

### Example 3: Dehydrochlorination of 250FB

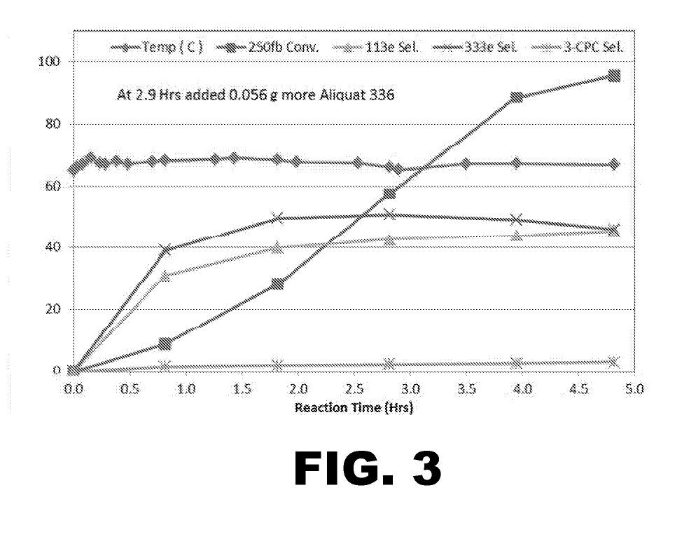

Example 1 was repeated using 14.2 g crude 250FB from a telomerization reaction of Tet and ethylene. Aliquat 336 was added at the start of the telomerization reaction in an amount of 0.058 g. The crude feed contained residual Tet, ethylene, by-product tetrachloropentanes, other minor by-products, Fe-TBP catalyst complex, and Aliquat 336. The concentration of 250fb was about 85 weight %. No Aliquat 336 was added initially to the dehydrochlorination reaction, other than the amount that was fed to the telomerization reaction. After 2.9 hours, 0.06 g of additional Aliquat 336 was added. The amount of NaOH/NaCl/H₂O solution was 6% molar excess of NaOH versus 250FB. FIG. 3 shows that conversion and selectivity similar to using purified 250FB feed were obtained. The impurities did not adversely affect the reaction. Further, the Aliquat 336 that was fed to the telomerization reaction was still active in the dehydrochlorination reaction.

1. A process for producing a chlorinated alkene, wherein the process comprises contacting
(a) a liquid chlorinated alkane stream comprising a soluble iron-tributylphosphate complex;
(b) an aqueous phase comprising an inorganic base;
(c) a phase transfer catalyst;
wherein the chlorinated alkene product has one less chlorine atom than the chlorinated alkane; and
wherein the chlorinated alkane stream contains high-boiling point chlorocarbons, catalysts, or combinations thereof; and is not further purified to remove these high boiling point components; and
wherein the aqueous phase comprising an inorganic base is produced from the chloroalkali process.

2. The process of claim 1, wherein the chlorinated alkane stream comprises 1,1,1,3-tetrachloropropane.

3. The process of claim 1, wherein the chlorinated alkene comprises 1,1,3-trichloropropene, 3,3,3-trichloropropene, 1,2,3-trichloropropene, or mixtures thereof.

4. (canceled)

5. (canceled)

6. The process of claim 1, wherein the metal portion of the soluble iron-tributylphosphate complex precipitates as a metal hydroxide; which is removed from the aqueous phase by filtration, settling, or centrifugation.

7. (canceled)

8. The process of claim 1, wherein the phase transfer catalyst comprises tetraalkylammonium compound, a tetraalkylphosphonium compound, a pyridinium salt, or combinations thereof.

9. The process of claim 8, wherein the phase transfer catalyst is selected from a group consisting of trioctylmethyl ammonium chloride (Aliquat 336), dioctyldimethylamonium chloride, Arquad 2HT-75, benzyldimethyldecylammonium chloride, benzyldimethyltetradecylammonium chloride, dimethyldioctadecylammonium chloride, dodecyltrimethylammonium chloride, methyltrioctylammonium chloride, tetrabutylammonium chloride, tetrahexylammonium chloride, tetraoctylammonium chloride, tridodecylmethylammonium chloride, tetramethylphosphonium chloride, tetraphenylphosphonium bromide, trihexyltetradecylphosphonium chloride, or combinations thereof.

10. The process of claim 9, wherein the phase transfer catalyst is trioctylmethylammonium chloride.

11. The process of claim 1, wherein the amount of phase transfer catalyst ranges from 0.01 to about 5.0 wt % based on the total weight of the components.

12. The process of claim 1, wherein the temperature of the process ranges from 45° C. to about 95° C.; and the pressure of the process ranges from 0 psig to about 200 psig.

13. (canceled)

14. The process of claim 1, wherein the organic phase from the dehydrochlorination reaction is further processed to remove the chlorinated alkene product(s) from the high-boiling chlorocarbons, catalysts, phase transfer catalyst, promoters, or combinations thereof.

15. The process of claim 1, wherein the organic phase from the dehydrochlorination reaction is further processed to remove the low boiling point components comprising carbon tetrachloride, ethylene, and water from the chlorinated alkene products(s).

16. The process of claim 1, wherein at least a portion of the organic phase comprising the high-boiling point chlorocarbons, catalysts, promoters, phase transfer catalysts, or combinations thereof are recycled to a reaction used to produce the chlorinated alkane starting material.

17. The process of claim 1, wherein the aqueous phase comprising an inorganic base further comprises up to a 26 wt % of a chloride salt selected from a group consisting of lithium chloride, sodium chloride, potassium chloride, barium chloride, calcium chloride, or combinations thereof.

18. The process of claim 17, wherein the chloride salt is sodium chloride.

19. The process of claim 1, wherein the mole ratio of the bases to the chlorinated alkane ranges from 0.1 to 2.0.

20. The process of claim 1, wherein the weight % of NaOH in the aqueous phase comprising an inorganic base is less than 20 wt %.

21. The process of claim 8, wherein the phase transfer catalyst is recycled to the dehydrochlorination reaction.

22. (canceled)

23. (canceled)

24. The process of claim 1, wherein the process is conducted in a jet stirred reactor or a series of jet stirred reactors.

25. The process of claim 1, wherein the process further comprises contacting the chlorinated alkene product with a chlorinating agent to produce a pentachloropropane; wherein the chlorinating agent comprises chlorine gas, sulfuryl chloride, or combinations thereof.

26. (canceled)

27. The process of claim 25, wherein the pentachloropropane is 1,1,1,2,3-pentachloropropane.

28. The process of claim 27, wherein the process further comprises contacting the pentachloropropane with at least one base, at least one catalyst, or combinations thereof to produce a tetrachloropropene.

29. The process of claim 28, wherein the tetrachloropropene is 1,1,2,3-tetrachloropropene.

30. (canceled)

31. (canceled)
